(19) United States
(12) Patent Application Publication
(10) Pub. No.: US 2013/0126559 A1
(43) Pub. Date: May 23, 2013

(54) Displacement Syringe

(71) Applicants: Kevin P. Cowan, Allison Park, PA (US); Mark Trocki, Cheswick, PA (US); Barry L. Tucker, Verona, PA (US); Edward J. Rhinehart, Monroeville, PA (US); Raymond C. Hoffman, Gibsonia, PA (US); David H. Berry, Kittanning, PA (US); Arthur E. Uber, III, Pittsburgh, PA (US)

(72) Inventors: Kevin P. Cowan, Allison Park, PA (US); Mark Trocki, Cheswick, PA (US); Barry L. Tucker, Verona, PA (US); Edward J. Rhinehart, Monroeville, PA (US); Raymond C. Hoffman, Gibsonia, PA (US); David H. Berry, Kittanning, PA (US); Arthur E. Uber, III, Pittsburgh, PA (US)

(73) Assignee: BAYER INTELLECTUAL PROPERTY GMBH, Monheim (DE)

(21) Appl. No.: 13/695,189

(22) PCT Filed: May 2, 2011
(86) PCT No.: PCT/US11/34792

§ 371 Date: Feb. 5, 2013

Related U.S. Application Data
(60) Provisional application No. 61/329,975, filed on Apr. 30, 2010.

(51) Int. Cl. B65D 88/54; G01F 11/00

(52) U.S. Cl. CPC B65D 83/0005
USPC 222/333; 222/386; 222/387

(57) ABSTRACT

A displacement syringe having a housing with a distal tip and a proximal end; a moveable outer plunger within the housing having an elongated passageway; at least one moveable inner plunger within the passageway of the outer plunger; at least one first seal between the outer plunger and the housing; and at least one second seal between the inner plunger and the outer plunger is disclosed herein. The syringe may have a lock at the proximal end of the housing and/or a valve at the distal tip of the housing. The syringe may also have a plurality of inner plungers of various diameters. A transfer system and delivery system are also disclosed herein. Methods to accurately and efficiently deliver small volumes using the displacement syringe are also disclosed.

### CROSS-REFERENCE TO RELATED APPLICATION

The present application claims priority to U.S. Provisional Patent Application No: 61/329,975 filed Apr. 30, 2010.

### FIELD OF THE DISCLOSURE

Embodiments of the present disclosure generally relate to displacement syringes.

### BACKGROUND

The following background information is provided to assist the reader to understand embodiments disclosed below and the environment in which they may be used. The terms used herein are not intended to be limited to any particular narrow interpretation unless clearly stated otherwise, either expressly or impliedly, in this document.

Syringes are used for delivering fluid, gel, and other slurries, including but not limited to cell slurries. It is to be understood that all future references to one or more of these types of substances, or “substances” generally, is not meant to be exclusive of the others when dispensing from one of the disclosed syringes is being discussed. For example, “fluid” may mean fluids, gels, or other slurries even though only one of these substances may be dispensed at a given time. Syringes, though, have limitations in accurately delivering very small amounts of substances due to issues related to size, volume, flow, manufacturing, seals, etc. In general, accuracy and delivery become more problematic as the size of the syringe increases, and particularly as the volume dispensed decreases. Current syringes can be ill-equipped to deliver a precise amount of a substance from a syringe at extremely low volumes, e.g., <100 microliter. Also, substances that require precise delivery at low quantities are often very expensive or delicate, such as pharmaceuticals or cell slurries. In addition, there is a need for a syringe capable of efficiently and accurately delivering multiple small amounts of a substance from a large volume or reservoir of the substance. As such, a syringe that is able to accomplish the following would be desirable: (i) accurately deliver very small amounts of a substance; (ii) automatically refill that substance within the syringe with each delivery of substance; (iii) automatically reset the syringe plunger; and/or (iv) completely empty the syringe with little to no residual substance remaining in the syringe. A syringe having one or more of these features may be useful for multiple applications, such as drug delivery, coating medical devices, such as balloon catheters and the like, cell culture techniques, dilutions, mixing, such as stem cell mixing, etc., and other applications requiring precise delivery of fluid, gels, or slurries as is known in the art.

### SUMMARY

In general, various embodiments of the present disclosure are directed to a displacement syringe. In an embodiment, the disclosed displacement syringe has a housing with a distal tip and a proximal end; a moveable outer plunger within the housing having an elongated passageway; at least one moveable inner plunger within the passageway of the outer plunger; at least one first seal between the outer plunger and the housing; and at least one second seal between the inner plunger and the outer plunger. The syringe may have a lock at the proximal end of the housing and/or a valve at the distal tip of the housing. The inner plunger may be a centerless ground rod, threaded screw, combination, or any profile with an exact volume. The syringe may have a plurality of inner plungers of various diameters. The inner plunger may be a smaller syringe. The inner plunger may be marked or have graduations to indicate volume. The syringe may have a push button, spring mechanism, piezoelectric stack, electroactive polymer, or motor to move the inner plunger. The syringe may have a flexible tip, an expandable bladder, or sterile fluid between the inner plunger and the outer plunger such that the inner plunger remains sterile by not contacting the substance to be dispensed and therefore, can be reused. The syringe may have a locking mechanism to lock the inner plunger and the outer plunger for large volume displacement.

In another embodiment, a delivery system is disclosed having a reservoir; a first valve located beneath the reservoir and connected to a housing with a closed end and an open end having a second valve to control dispensing; and at least one plunger enclosed in the housing with at least one seal to prevent leaking. The plunger may be a centerless ground rod, a threaded screw, a combination, or any profile with an exact volume. The system may have a push button, spring mechanism, piezoelectric stack, electroactive polymer, or motor to move the plunger.

In another embodiment, a transfer system is disclosed having a first syringe with a lock at the distal tip and a second syringe with a distal valve and a plunger having a reflux valve and a lock that connects with the lock on the first syringe. The first syringe may have a plunger that is a centerless ground rod, threaded screw, combination, or any profile with an exact volume as embodied herein. The first syringe may contain a concentrate, such as stem cells, and the second syringe may contain a dilutant, such as buffered solution, such that the system may be utilized for mixing and dispensing.

These and other details, objects, and advantages of the present disclosure will become better understood or apparent from the following description and drawings showing embodiments thereof.

### BRIEF DESCRIPTION OF THE DRAWINGS

The accompanying drawings illustrate examples of embodiments of the disclosure. In such drawings:

FIG. 1 shows a diagram of a syringe having an inner displacement plunger and an outer large volume plunger according to various embodiments of the present disclosure;

FIG. 2 shows a diagram of a displacement syringe with a volume conserving tip, valve, and one way ratchet lock with release mechanism according to various embodiments of the present disclosure;

FIG. 3 shows a diagram of a displacement syringe with a threaded screw type inner plunger and a motor according to various embodiments of the present disclosure;

FIG. 4 shows a diagram of a displacement syringe with multiple inner plungers according to various embodiments of the present disclosure;

FIG. 5 shows a diagram of a displacement syringe with a stop on the inner plunger near the proximal end according to various embodiments of the present disclosure;

FIG. 6 shows a diagram of a displacement syringe with an inner plunger that does not extend beyond an outer plunger and the shape of the outer plunger corresponds to the shape of the housing according to various embodiments of the present disclosure;

FIG. 7 shows a diagram of a displacement syringe with the second seal near the proximal end according to various embodiments of the present disclosure;

FIG. 8 shows a diagram of a displacement syringe with a fixed outer plunger and a roller on the housing according to various embodiments of the present disclosure;

FIGS. 9A-9C show diagrams of a wedge between the outer plunger and the housing of a displacement syringe according to various embodiments of the present disclosure;

FIG. 10 shows a diagram of a displacement syringe with fluid being displaced through electroactive polymers enclosed within a bladder (10A) or in direct contact with the fluid to be dispensed and expanded (10B) according to various embodiments of the present disclosure;

FIG. 11 shows a diagram of the inner plunger having a lock and/or a stop at the distal end for securing within the outer plunger according to various embodiments of the present disclosure;

FIG. 12 shows a diagram of a displacement syringe with a flexible tip on the distal end of the outer plunger according to various embodiments of the present disclosure;

FIG. 13 shows a diagram of a displacement syringe with an expandable bladder on the proximal end of the outer plunger according to various embodiments of the present disclosure;

FIG. 14 shows a diagram of a displacement syringe with fluid between the inner plunger and the outer plunger according to various embodiments of the present disclosure;

FIG. 15 shows a diagram of a displacement syringe with fluid between the inner plunger and the outer plunger with the outer plunger controlling movement of a smaller syringe according to various embodiments of the present disclosure;

FIG. 16 shows a diagram of a fluid delivery system having a reservoir connected to a perpendicular plunger according to various embodiments of the present disclosure;

FIG. 17 shows a diagram of a displacement syringe having a push button actuation mechanism according to various embodiments of the present disclosure; and

FIG. 18 shows a diagram of a fluid transfer system with interlocking syringes according to various embodiments of the present disclosure.

### DESCRIPTION

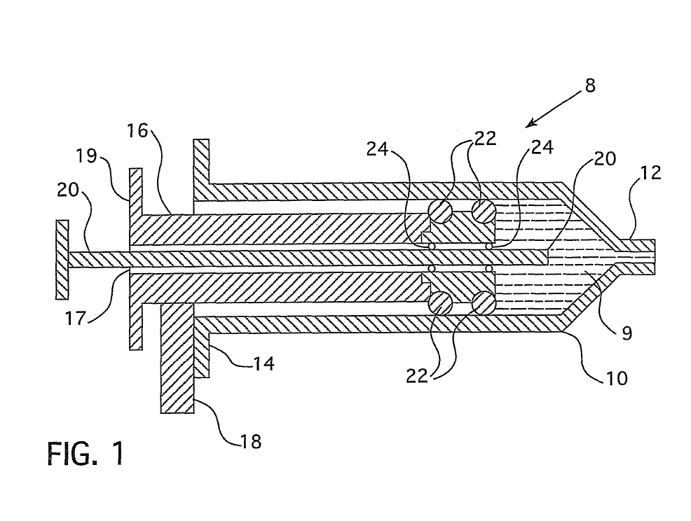

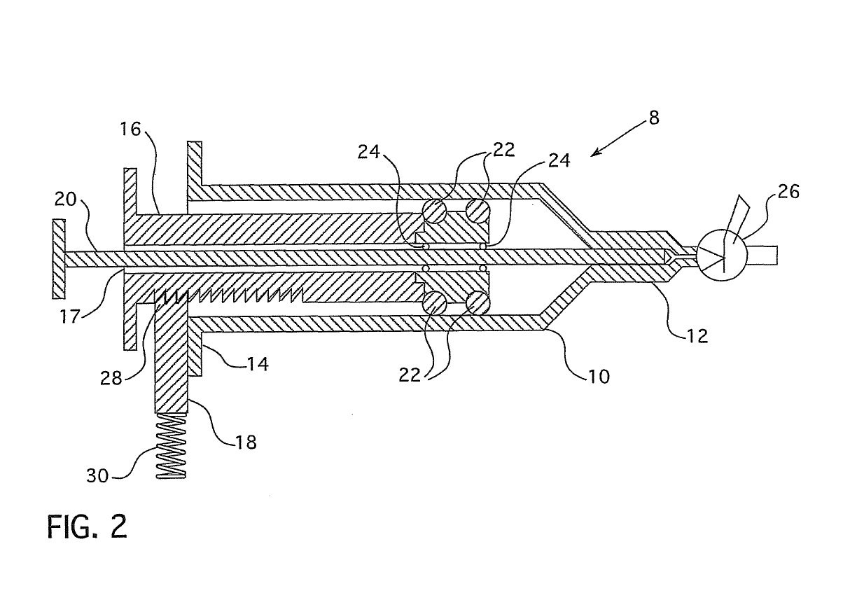

In all of its embodiments, the present disclosure relates to a displacement syringe 8 that is capable of accurately dispensing either very small amounts of a substance 9 or large volume amounts of that same substance 9. In an embodiment, as shown in FIG. 1, the displacement syringe 8 has a housing 10 with a distal tip 12 and a proximal end 14; a moveable outer plunger 16 within the housing 10 having an elongated passageway 17; at least one moveable inner plunger 20 within the passageway 17 of the outer plunger 16; at least one first seal 22 between the outer plunger 16 and the housing 10; and at least one second seal 24 between the inner plunger 20 and the outer plunger 16. The seals 22, 24 prevent substances from exiting the elongated passageway 17 or the housing 10. The displacement syringe 8 may have a locking mechanism 18 to lock the outer plunger 16 into position while allowing for movement of the inner plunger 20. In an example, as illustrated in FIG. 1, the locking mechanism 18 may be located at the proximal end 14 of the housing 10. In an alternate embodiment, the outer plunger 16 may be locked to the housing 10 or the locking mechanism may be disengaged by manually twisting the outer plunger 16. As illustrated in FIG. 2, the displacement syringe 8 may also have a valve 26 at the distal tip 12 of the housing 10 to control fluid dispensing. The valve 26 may be, for example, a one-way check valve, a stopcock, or any other suitable automated or manually operated valve.

For any of the embodiments disclosed herein, the housing 10 of the syringe 8 may be constructed of, for example, glass, plastic, polypropylene, polycarbonate, etc. or any material that is capable of holding a volume of fluid or other substance. The housing 10 may be marked to indicate volume in, for example, microliters or other volumetric indicators. In an example, the housing 10 is a Hamilton syringe. As illustrated in FIG. 2, the distal tip 12 of the housing 10 may be a volume conserving tip. The outer plunger 16 for any of the embodiments disclosed within may be constructed of, for example, plastic, polypropylene, polyethylene, etc. The shape of the outer plunger 16 may match the profile of the housing 10 of the syringe 8. The outer plunger 16 may also be marked to indicate volume delivered in, for example, microliters and the like.

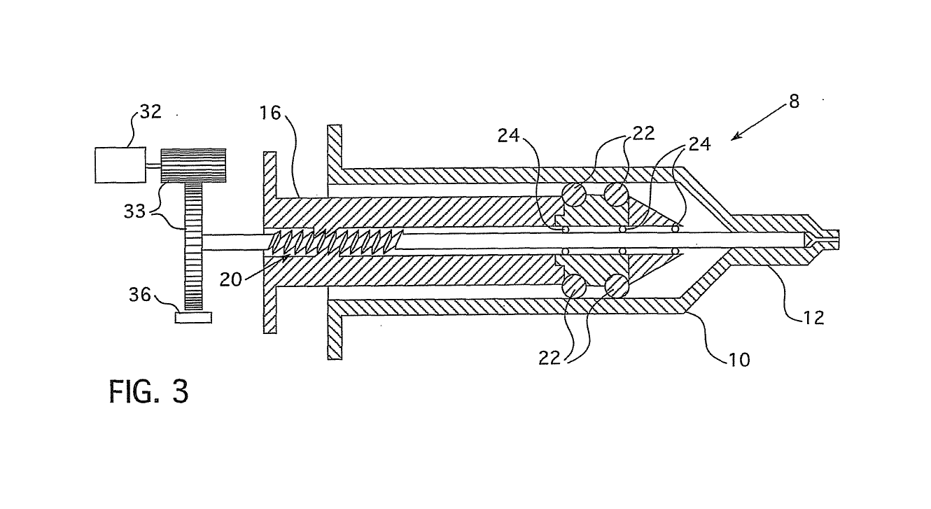

The inner plunger 20 of any of the embodiments herein disclosed may be one of a rod (FIGS. 1 and 2), a threaded screw (not shown), or a combination (FIG. 3), or any profile with an exact volume, for example a centerless ground rod with a precisely controlled diameter. The inner plunger 20 may be constructed of, for example, stainless steel, glass, ceramic, Teflon® polytetrafluoroethylene, plastic, loaded plastic, such as plastic mixed with at least one other material, etc. and made by, for example, precision grinding, centerless grinding, injection molding, extrusion, etc. As illustrated in FIGS. 2 and 3, the inner plunger 20 may have a diameter to fit within the distal tip 12 and/or shaped to match the profile of the distal tip 12, for example a volume conserving tip. In another example, the inner plunger 20 may be stepped or tapered. In such an embodiment, the entire volume in the housing 10 can be delivered during operation. In all embodiments, because the substance within the housing 10 is at vacuum pressure, delivery of the fluid or substance, no matter how small, will cause the outer plunger 16 to gradually move towards the distal tip 12 as the syringe empties. The inner plunger 20 may move inside the outer plunger 16, inside the housing 10, and/or into the distal tip 12 of the housing 10. In an example and without limitation, the housing 10 of the syringe 8 may hold a volume in the range of about 1-50 ml and the inner plunger 20 may accurately dispense through axial displacement a volume in the range of about 0.7- 120 μl. The precise volume of fluid 9 displaced is a function of the travel length of the inner plunger 20 times the area of the inner plunger 20; therefore, the total volume of fluid 9 displaced is limited by the diameter and distance traveled of the inner plunger 20 as well as the remaining length in the housing 10 of the syringe 8.

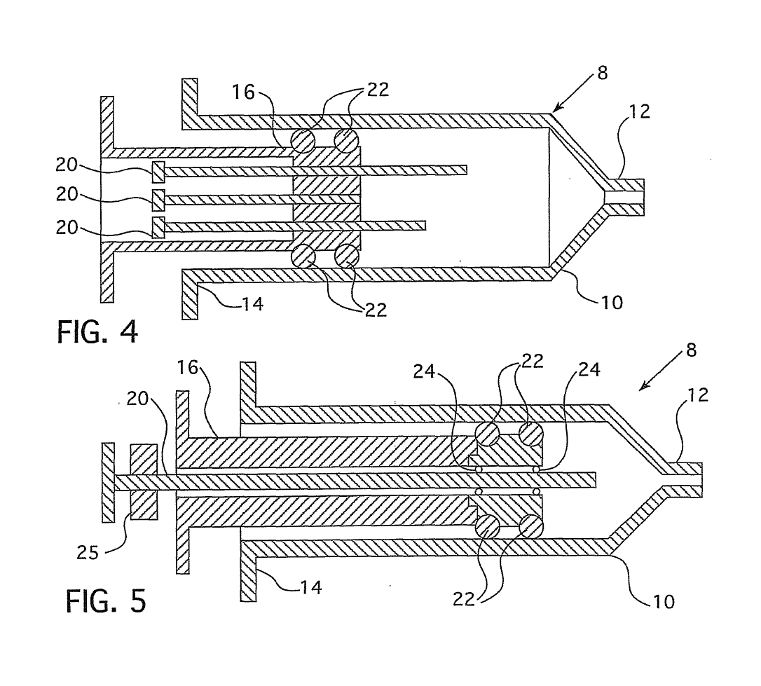

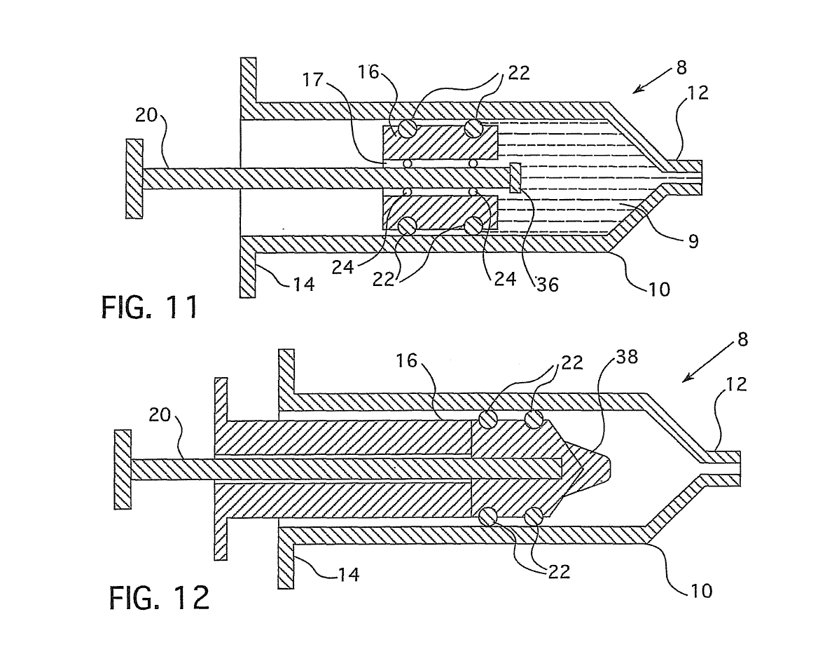

The inner plunger 20 may be concentric within the outer plunger 16. In various embodiments, as illustrated in FIG. 4, the displacement syringe 8 may include multiple inner plungers 20 of the same or varying diameters to accurately control various doses with a single displacement syringe 8. The multiple inner plungers 20 may be, for example, coaxial (not shown). In an example, one of the inner plungers 20 may be a specific length and diameter to precisely displace 10 microliters and another inner plunger designed to precisely displace 30 microliters, such that 10, 30 or 40 microliters could be dispensed. Each of the inner plungers 20 may be pulled back and reset. Each inner plunger 20 may have at least one seal 24 or the syringe 8 may have one seal with multiple holes corresponding to the inner plungers 20. The inner plungers may have stops 25 and 36 at the proximal and/or distal end as illustrated in FIGS. 5 and 11.

As illustrated in FIG. 5, the inner plunger 20 may have a stop 25 near the proximal end 14 for repeatable dose volumes. The inner plunger may have a spring (not shown) between the proximal end 14 and the stop 25. By using the stop 25, with or without a spring, a consistent amount of volume can be delivered as the outer plunger 16 moves closer to the distal tip 12 with each delivery of fluid or other substance.

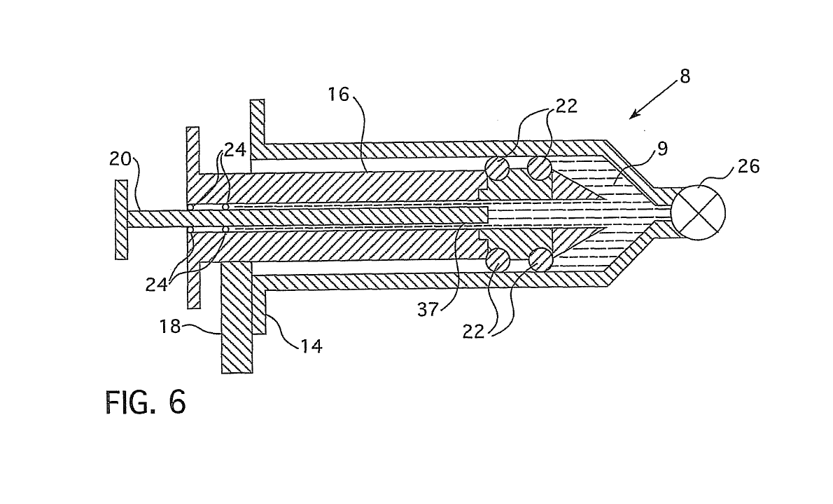

As illustrated in FIG. 6, the inner plunger 20 may not move beyond the outer plunger 16. A gap 37 between the inner plunger 20 and the outer plunger 16 allows the inner plunger 20 to displace fluid 9 without extending beyond the tip of the outer plunger 16. The seals 24 located near the proximal end 14 prevent fluid 9 from exiting the proximal end 14 of the syringe 8. The outer plunger 16 may be shaped to correspond to the shape of the housing 10 so that all of the fluid 9 may be dispensed.

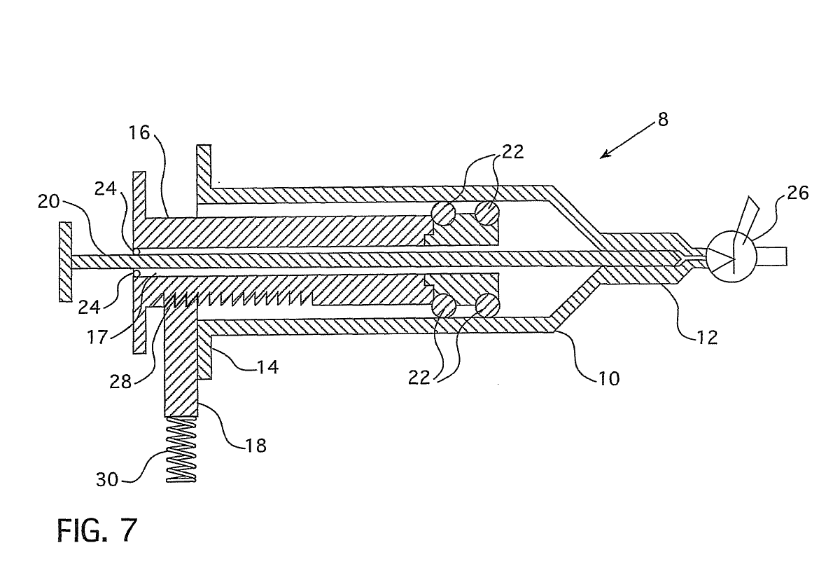

The first seal 22 and the second seal 24, as illustrated in FIGS. 1 and 2, allow the outer plunger 16 to advance under the vacuum created by retracting the inner plunger 20. In any of the embodiments disclosed herein the first seal 22 and the second seal 24 may be constructed from any suitable material such as, for example without limitation, Buna-n, rubber, ethylene propylene diene monomer (EPDM), precision ground graphite, Teflon® polytetrafluoroethylene, coated silicone elastomer such as chemically bonded or coated parylene, etc. For example, the seals 22 and 24 are at least one O-ring seal. Having more than one O-ring, for example two, as shown in FIGS. 1 and 2, may provide increased stability. In another example, the seals 22 and 24 are formed from one rubber cover with a hole for the inner plunger 20. In yet another example, as illustrated in FIG. 7, the second seal 24 may be near the proximal end of the outer plunger 16. In an alternate example, in addition to at least the first seal 22, the passageway of the outer plunger 16 may have a smaller diameter at the proximal end 14 such that there is a tight tolerance or fit for the inner plunger 16, thereby increasing stability.

As illustrated in FIGS. 1 and 2, the lock 18 at the proximal end 14 of the housing 10 may be any device to hold the outer plunger 16 in position relative to the housing 10 to prevent the outer plunger 16 from moving back. For example, the lock 18 may be a clamp, a one-way ratchet with release mechanism (FIG. 2), a push button lock, a lock and pin, etc. In the example of the one-way ratchet with a release mechanism type lock illustrated in FIG. 2, the lock 18 is attached to the housing 10 and the outer plunger 16 has teeth 28 corresponding to the lock 18. In such an embodiment, the outer plunger 16 may have to be pulled backward slightly to secure the outer plunger 16 into position. The lock 18 may alternatively have a spring loaded bias 30.

In another example, the housing 10 may be mounted in place and the outer plunger 16 may be secured by a locking device, such as a pin, which locks into the mounting device or an adaptor plate (not shown). The locking device, in the form of a pin, may be adapted to fit into one or more holes in the outer plunger 16 and one or more holes in the housing 10. In operation, the locking device secures the outer plunger 16 to the housing 10, and the inner plunger 20 could then be advanced for accurate delivery of fluid 9. The inner plunger 20 may be advanced until it has no travel room, at which time the valve 26 could be closed. The locking device may then be disengaged from the outer plunger 16 and the outer plunger 16 may be advanced, which causes the inner plunger 20 to be pushed back. The outer plunger 16 is advanced until a hole in the outer plunger 16 is lined up with a hole in the housing 10, and the locking device may be engaged. The valve 26 may then be opened and fluid 9 again delivered by advancing the inner plunger 20.

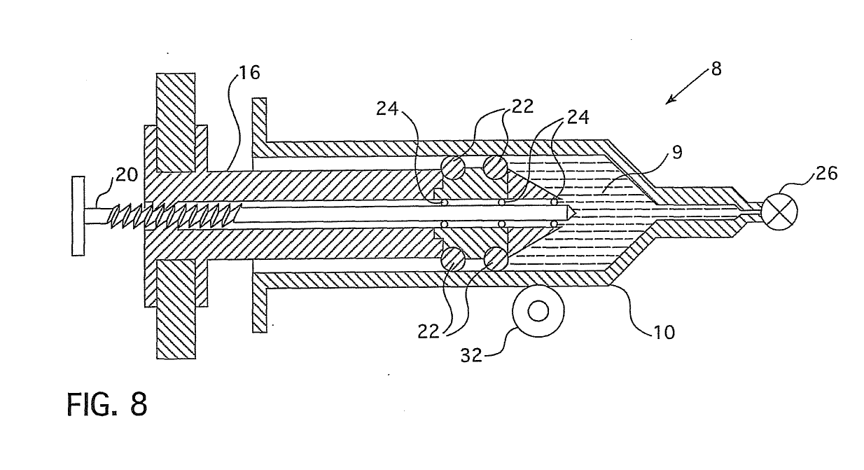

As illustrated in FIG. 8, the housing 10 may be on a roller 32 to allow movement. In such embodiment, the outer plunger 16 is fixed in place and during fluid 9 delivery the housing 10 is held in place by friction when the inner plunger 20 is advanced. When the valve 26 is closed and the inner plunger 20 is withdrawn, a vacuum force draws the housing 10 back toward the outer plunger 16.

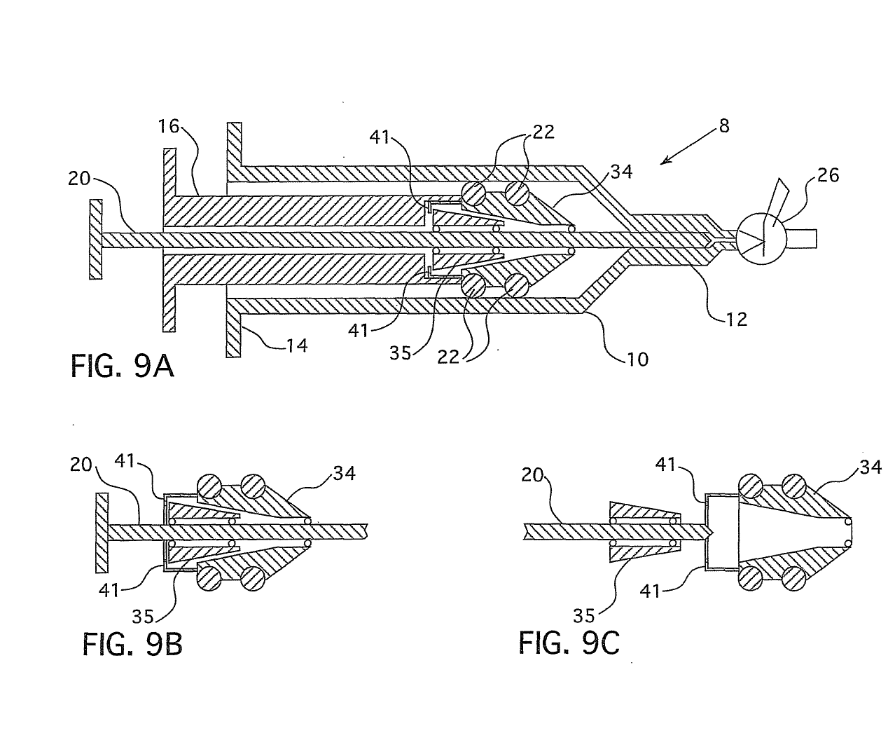

In alternate embodiments illustrated in FIGS. 9A, 9B and 9C, instead of the lock 18, the syringe 8 has a one-way locking mechanism, such as wedges 34, 35, that lock forward but release in reverse. In operation, as the inner plunger 20 moves forward, the wedge 34 is pulled forward due to the force of friction. As the wedge 34 moves, the wedges 34, 35 slide and create a radial locking force. The syringe 8 illustrated in FIGS. 9A, 9B and 9C includes a stop 41 that does not allow the wedge 35 to move rearward more than a preset distance from the wedge 34.

The valve 26 controls fluid dispensing from the distal tip 12. For example, as illustrated in FIGS. 2 and 7, the valve 26 is a manual or automatic stopcock, one-way check valve, manual or automatic open/close valve, etc.

In an embodiment, in operation the housing 10 of the syringe 8 is filled with a volume of fluid 9 by pulling back both the inner plunger 20 and the outer plunger 16. Then the outer plunger 16 is locked to the housing 10. The inner plunger 20 may be advanced with accuracy to deliver very small doses until the inner plunger 20 bottoms out at the distal tip 12 of the housing 10. The fluid 9 dispensed out of the distal end 12 is equal to the volume displaced by the precisely controlled inner plunger 20. The dispensing accuracy is independent of the size, shape, and tolerance of the housing 10. To reset the inner plunger 20, the valve 26 may be activated or closed at the distal end 12 of the housing 10 and the lock 18 disengaged. By pushing on the outer plunger 16, the inner plunger 20 is driven back by pressure. Alternatively, by pulling the inner plunger 20 back, the outer plunger 16 is advanced by lower pressure created in the fluid 9. When the inner plunger 20 is back to the desired position, the lock 18 is engaged and the valve 26 is opened. The inner plunger 20 may then be advanced for accurate fluid 9 dispensing. In this embodiment, multiple small doses of very low quantities may be dispensed from the larger volume in the housing 10 of the syringe 8. By cycling the precisely controlled inner plunger 20 with the outer plunger 16, the full volume in the housing 10 of the syringe 8 may be delivered.

The inner plunger 20 may be actively and precisely controlled by hand, for example by push button or a spring mechanism, or, as illustrated in FIG. 3, by a motor 32, such as a linear motor or a piezoelectric motor. In examples, the inner plunger 20 may be pushed forward or screwed. In another example, the inner plunger 20 may be graduated for a specific volume or the inner plunger 20 may be clear and marked with graduations. In one embodiment, the markings on the inner plunger 20 may be configured such that an edge 19 (FIG. 1) of the outer plunger 16 may be used as a reference guide as to how many or which markings on the inner plunger 20 have passed the edge 19 of the outer plunger 16. In a further example, the inner plunger 20 may be both graduated and have a push button used to reset the system each time. In yet another example, as shown in FIG. 3, the motor 32 may be used with a spline 33 and an encoder reader 36 or counter to control the number of revolutions for a screw type inner plunger 20.

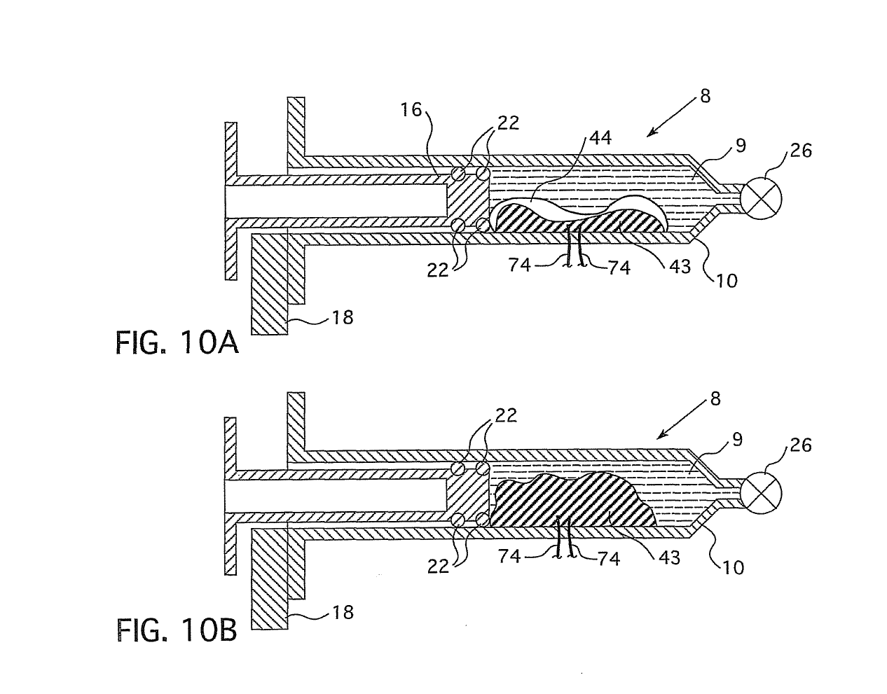

Other examples for controlling movement of the inner plunger 20 include piezoelectric stacks, electroactive polymers, and wax or polymer expansion materials. Piezoelectric stacks or electroactive polymers will expand a given amount for a known voltage or current. As the piezoelectric stacks or electroactive polymers expand the inner plunger 20 moves a precise distance thereby displacing the fluid 9. In an alternate embodiment illustrated in FIG. 10, the piezoelectric stacks or electroactive polymer 43 may be in fluid contact with the fluid 9 to be dispensed or enclosed within a bladder 44 (FIG. 10A) and as the polymer expands a precise amount of fluid would be dispensed (FIG. 10B). The electroactive polymer 43 is attached to wires 74 connecting the electroactive polymer 43 to the voltage source (not shown). The wires 74 may be connected directly through the housing (FIG. 10) or at any position that allows the electroactive polymers 43 access to the current or voltage. The valve 26 is opened and a known voltage is applied to expand the electroactive polymer 43 thereby dispensing the desired amount of fluid or substance 9. Then, the valve 26 is closed, the lock 18 disengaged, and the voltage removed so that the electroactive polymer 43 relaxes, such that a solid plunger 16 can reset for further dispensing.

In an alternate embodiment, with the lock 18 disengaged and the valve 26 open, a large volume of the substance 9 can be dispensed by moving both the outer plunger 16 and the inner plunger 20 toward the distal end 12. In an example, a locking mechanism may be used to lock the inner plunger 20 with the outer plunger 16, thereby allowing movement of both the inner plunger 20 and the outer plunger 16. As illustrated in FIG. 11, the inner plunger 20 may have a stop 36 on the distal end to lock the inner plunger 20 to the outer plunger 16 for retracting, filling, and/or large volume delivery. The inner plunger 20 or the stop 36 may also lock the outer plunger 16 to the housing 10 by expanding the outer plunger 16, thereby increasing the friction between seal 22 and the housing 10. For example, the inner plunger 20 and/or the stop 36 may be shaped, such as oval shaped, such that as the inner plunger 20 and/or the stop 36 is twisted into the similarly shaped passageway 17 of the outer plunger 16, the outer plunger 16 expands and locks to the housing 10 through increased friction. When the shapes of the inner plunger 20 and/or the stop 36 are aligned with the passageway 17, the inner plunger 20 and/or the stop 36 move within the passageway 17. In another example, the inner plunger 20 may expand or lock the outer plunger 16 by any mechanism, such as a lock and pin. In an alternate embodiment, also illustrated in FIG. 11, the outer plunger 16 may not extend beyond the proximal end 14 of the housing 10. By locking the inner plunger 20 to the outer plunger 16 as described above, the outer plunger 16 can be pushed forward or retracted with the inner plunger 20 as needed.

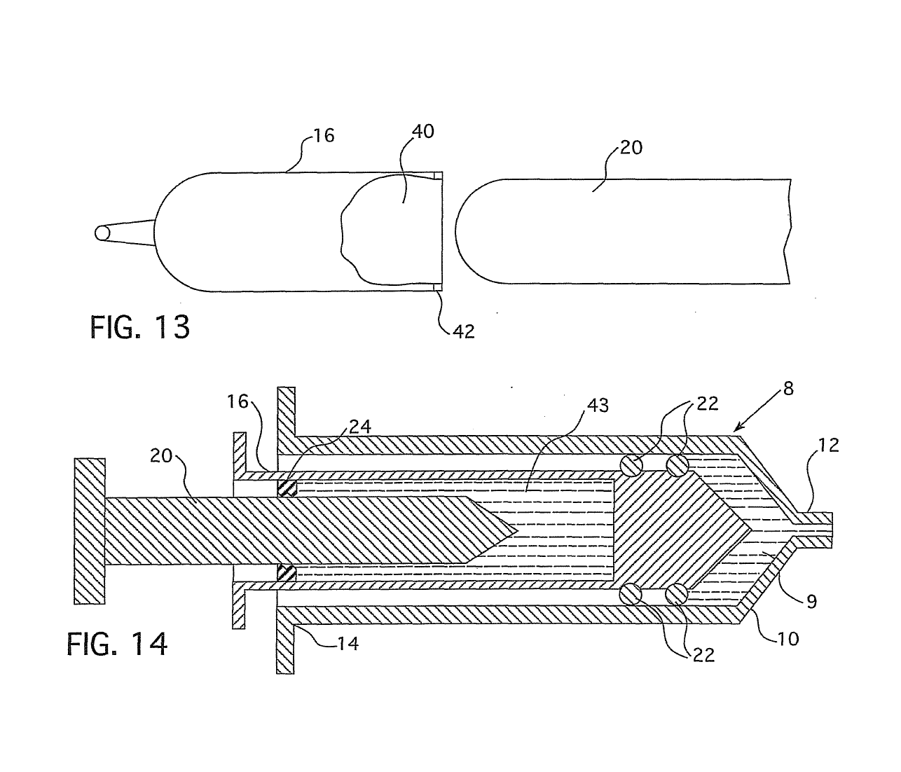

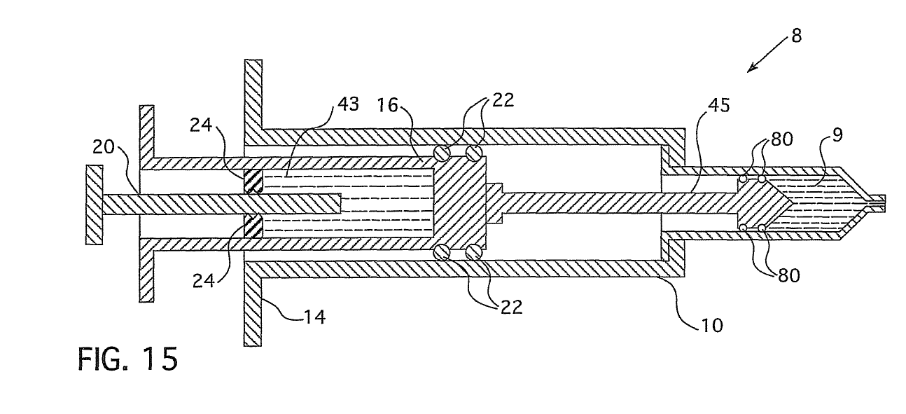

Embodiments which would allow the inner plunger 20 to remain sterile and not come in contact with the fluid 9 to be dispensed such that it could be reused include an expandable flexible tip 38 at the distal end of the outer plunger 20 (FIG. 12) or an expandable bladder 40 at the proximal end of the outer plunger 20 (FIG. 13). The inner plunger 20 pushes against the flexible tip 38 or the bladder 40 to dispense the displaced volume of fluid 9. The bladder 40 is snapped into the proximal end of the outer plunger 16 with a retainer ring 42. The flexible tip 38 or the bladder 40 does not permit the fluid 9 to be dispensed from contacting the inner plunger 20 so that the inner plunger 20 may be reused without contamination, and thereby may reduce cost. In an alternate embodiment, as illustrated in FIG. 14, the inner plunger 20 may be pushed into an incompressible sterile fluid 43, such as saline, which drives the outer plunger 16 forward to displace the fluid 9 to be dispersed. The fluid 43 isolates the inner plunger 20 from the fluid or substance 9 to be dispersed such that the inner plunger 20 can be reused. In another alternate embodiment, as illustrated in FIG. 15, the inner plunger 20 may be pushed into an incompressible sterile fluid 43, such as saline, which drives the plunger 45 of another syringe to displace a precise amount of fluid 9 to be dispersed. In this embodiment, the volume of fluid 9 dispensed is a function of the travel length of the inner plunger 20 times the area of the inner plunger 20 and the ratio of the diameters of the plungers 16 and 45. If the plunger 45 is smaller than the outer plunger 16, then the amount of fluid displaced would be reduced by the ratio of the diameters of the plungers 16 and 45 and the resolution would be increased.

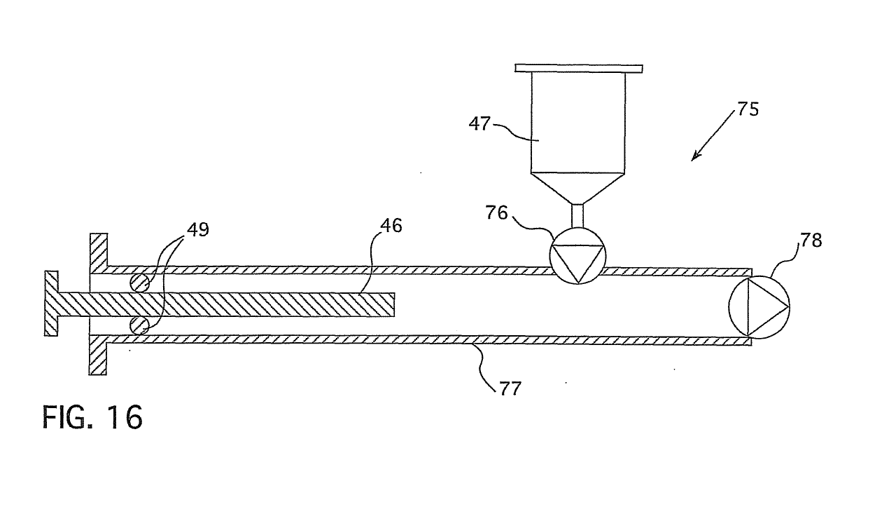

In another embodiment, as illustrated in FIG. 16, a delivery system 75 may have a reservoir or large container 47 having a first control valve 76 that dispenses fluid into a housing 77 with a closed end, an open end with a second control valve 78 to control fluid dispensing and at least one plunger 46, such as the inner plunger 20 described herein for dispensing a precise amount of fluid through valve 78 at the open end. The control valves 76, 78 may be, for example and without limitation, one-way check valves. A seal 49, such as an O-ring seal, prevents fluid from exiting the housing around the plunger 46. As the plunger 46 is advanced, a precise amount of fluid or substance is dispensed through valve 78. The plunger 46 may be located at any position within the housing 77 allowing enough travel length to displace a precise volume of fluid. The housing 77 may be any shape that provides enough travel length for the plunger 46 to displace a precise amount of fluid. In an example, the plunger 46 is perpendicular to the reservoir 47 as illustrated in FIG. 16. To reset the system, the reverse flow through 78 is restricted, the plunger 46 is drawn back, and fluid is supplied from the container 47. The plunger 46 may be controlled manually, by a motor, such as a linear motor or piezoelectric motor, by piezoelectric stacks, or any other means as described above herein. Such control of plunger 46, on this and other embodiments disclosed herein, provide for precise, repeatable delivery of the same amount of fluid as used, for example, in a production line. Through the automatic refill of the devices disclosed in FIG. 16, an entire reservoir 47 of fluid can be automatically delivered in small, repeated doses without the need for manual intervention.

In any of the embodiments disclosed herein, if a sample of the fluid or substance to be dispensed from the delivery system 75 (FIG. 16) or the syringe 8 (FIG. 2) described herein is needed, without it being registered as a delivered amount through displacement, the syringe 8 or delivery system 75 may have a sampling valve (not shown) with access to the substance. For example, the syringe 8 (FIG. 2) may have a sampling valve near the distal tip and the delivery system 75 (FIG. 16) may have a sampling valve near the open end or on the reservoir 47. In examples, the sampling valve may be a reflux valve, T-connector, or three-way stop cock. In an example, to take a sample from syringe 8 illustrated in FIG. 2, the valve 26 is closed, the lock 18 disengaged (if applicable), the sample taken by opening and closing the sampling valve (not shown), and then the outlet valve 26 opened so that the system resets.

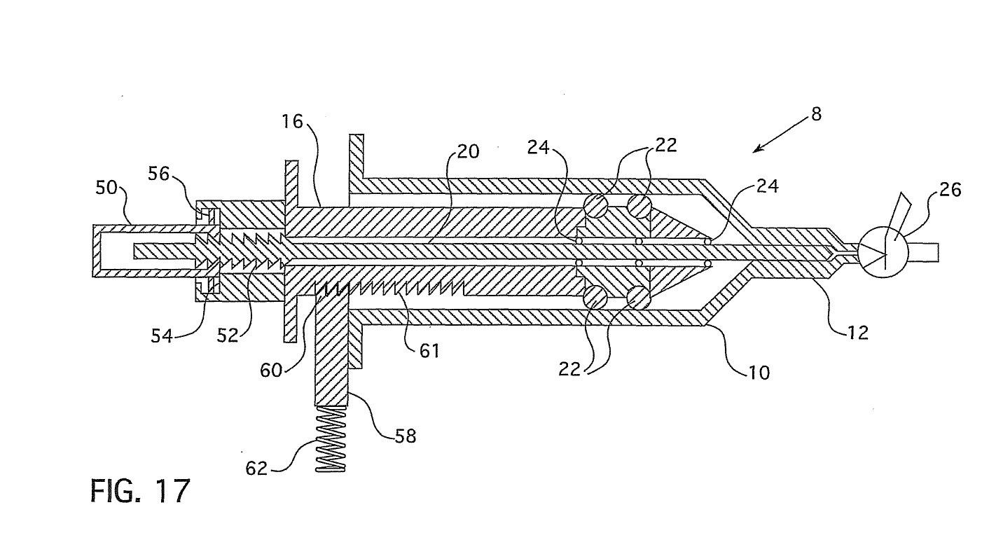

In one embodiment as illustrated in FIG. 17, the displacement syringe 8 includes a push button 50 that advances the inner plunger 20 a predetermined distance, so that a predetermined amount of fluid is dispensed each time the push button 50 is manually or automatically actuated (i.e., depressed). The inner plunger 20 includes ratchet teeth 52 that are engaged by the push button 50. The outer plunger 16 includes a stop 54 that prohibits the push button 50 from advancing beyond a predetermined distance when the push button 50 is depressed. A rear stop 56 prohibits the push button 50 from exiting the outer plunger 16.

In one embodiment illustrated in FIG. 17, the shape of the forward end of the inner plunger 16 matches the profile of the housing 10. Also, in one embodiment as illustrated in FIG. 17, the syringe 8 includes a plunger lock 58 on the housing 10 that has ratchet teeth 60 that engage with ratchet teeth 61 on the outer plunger 16. A spring 62 biases the lock 58 toward the outer plunger 16 such that the ratchet teeth 60, 61 are normally engaged to prohibit backward movement of the outer plunger 16 absent downward movement of the lock 58, while allowing forward movement of the outer plunger 16.

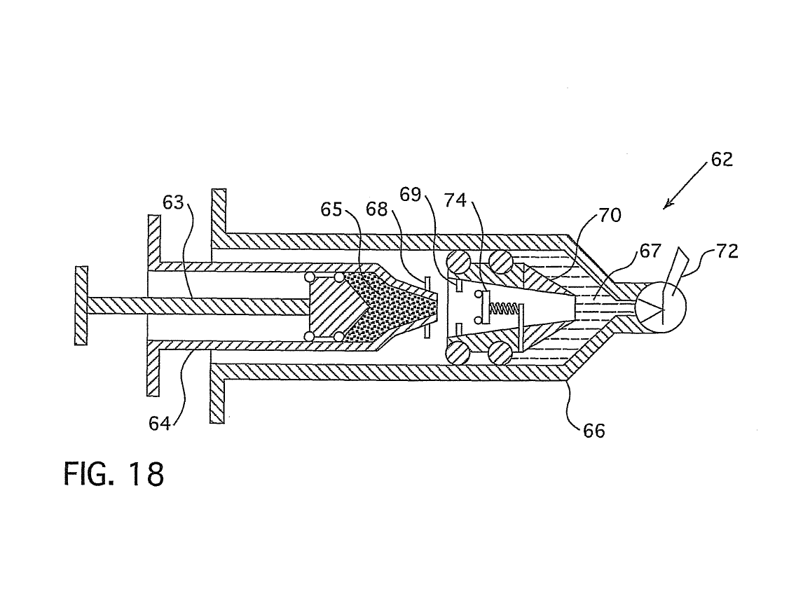

In another embodiment, as illustrated in FIG. 18, a transfer system 62 is disclosed with interlocking syringes for mixing substances, cells, etc. A syringe 64 would be prepared with a concentrate 65, for example and without limitation, cells, such as stem cells, drug, etc. A syringe 66 would be prepared with a dilutant 67, such as saline, buffered solution, cell culture media, etc. After the syringes 64 and 66 are prepared, they are connected, for example by a male luer lock 68 on syringe 64 connected to a female luer lock 69 on syringe 66. Through connection, a reflux valve 74 on the plunger 70 of syringe 66 is opened and a distal valve 72, such as a stopcock or one-way check valve, at the distal tip 73 of syringe 66 is closed. Then, by pushing and pulling the plunger 63 of syringe 64, the substances 65 and 67 in the syringes 64 and 66, respectively, are mixed. The mixture is delivered by advancing plunger 63 to the end of syringe 64, opening the valve 72, and then advancing the plunger 70 of syringe 66 by pushing the syringe 64 forward. In embodiments, the syringe 66 may be pre-loaded prior to use, loaded through valve 72, or loaded using syringe 64 after it is connected to syringe 66. In an alternate embodiment, the plunger 63 of syringe 64 may be the inner plunger 20 described herein above, for example a centerless ground rod. After mixing, the inner plunger 20 is retracted, the valve 72 opened, and a precise amount of the mixture is delivered by advancing the inner plunger 20.

The displacement syringe 8 and systems 62, 75 disclosed herein provide the ability to use one package or system to deliver multiple doses while limiting waste of reagents and saving time and resources. The embodiments disclosed herein may be used for, for example, drug delivery, coating medical devices, such as for example and without limitation balloon catheters, cell culture techniques, dilutions, mixing, etc.

### EXAMPLE

The following discussion illustrates non-limiting examples of embodiments of the present disclosure.

A 5 ml plastic displacement syringe 2.5 inches long with a 0.43 inch inside diameter, a 3.5 inches long polypropylene outer plunger, and a 4.2 inches long screw type inner plunger, as described herein, having a 0.15 inch diameter and a movement of 0.31 inch/revolution displaces 9.4 μl/revolution. Similarly, a rod type inner plunger displaces 12 μl/mm of rod moved.

The present disclosure has been described with reference to specific details of particular embodiments thereof. It is not intended that such details be regarded as limitations upon the scope of the disclosure except insofar as and to the extent that they are included in the accompanying claims.

What is claimed is:

1. A syringe comprising:
a housing having a distal tip and a proximal end;
a moveable outer plunger within the housing having an elongated passageway;
at least one moveable inner plunger within the passageway of the outer plunger;
at least one first seal between the outer plunger and the housing; and
at least one second seal between the inner plunger and the outer plunger.

2. The syringe of claim 1, further comprising a locking mechanism.

3. The syringe of claim 1, further comprising a a valve at the distal tip of the housing.

4. (canceled)

5. (canceled)

6. (canceled)

7. The syringe of claim 1, wherein the inner plunger is a centerless ground rod, threaded screw, or combination.

8. (canceled)

9. The syringe of claim 1, further comprising a plurality of inner plungers with various diameters.

10. (canceled)

11. (canceled)

12. (canceled)

13. The syringe of claim 1, wherein the inner plunger is moveable within the outer plunger, inside the housing, or into the distal tip.

14. (canceled)

15. (canceled)

16. The syringe of claim 1, wherein the inner plunger is stepped or tapered.

17. (canceled)

18. (canceled)

19. (canceled)

20. The syringe of claim 1, wherein the first seal and the second seal are located near the distal tip.

21. The syringe of claim 1, wherein the second seal is located near the proximal end.

22. (canceled)

23. (canceled)

24. (canceled)

25. (canceled)

26. The syringe of claim 1, further comprising a push button, spring mechanism, piezoelectric stack, electroactive polymer, or motor to move the inner plunger.

27. (canceled)

28. (canceled)

29. (canceled)

30. The syringe of claim 1, further comprising a locking mechanism to lock the inner plunger with the outer plunger.

31. The syringe of claim 1, further comprising a stop on the inner plunger at the distal end.

32. The syringe of claim 1, further comprising a flexible tip at the distal end of the outer plunger.

33. The syringe of claim 1, further comprising an expandable bladder at the proximal end of the outer plunger.

34. (canceled)

35. The syringe of claim 1, further comprising a roller connected to the housing.

36. A delivery system comprising a reservoir, a first valve located beneath the reservoir and connected to a housing having a closed end and an open end with a second valve, and at least one plunger enclosed in the housing and having at least one seal.

37. The delivery system of claim 36, wherein the plunger is a centerless ground rod, threaded screw, combination or any profile with an exact volume.

38. (canceled)

39. (canceled)

40. A transfer system comprising a first syringe having a lock at the distal tip and a second syringe having a distal valve and a plunger with a reflux valve and a lock that connects with the lock of the first syringe.

41. (canceled)

42. The transfer system of claim 40, wherein the first syringe contains a concentrate.

43. The transfer system of claim 40, wherein the second syringe contains a dilutant.
